SEQUENCE LISTING (2 sequences)

SEQ ID NO: 1 - DNA, 19 bp, Artificial Sequence
  Primer 338F
actcctacgggaggcagca

SEQ ID NO: 2 - DNA, 20 bp, Artificial Sequence
  Primer 806R
ggactachvgggtwtctaat

(19) United States
(12) Patent Application Publication
(10) Pub. No.: US 2022/0135936 A1
(43) Pub. Date: May 5, 2022

(54) BREVUNDIMONAS STRAIN AS ENDOSYMBIONT OF VERTICILLIUM DAHLIAE AND USE THEREOF

(71) Applicant: Tarim University, Ala'er City (CN)

(72) Inventors: Hong ZENG, Ala'er City (CN); Chang GAO, Ala'er City (CN); Lingqi HUA, Ala'er City (CN); Guimin LIU, Ala'er City (CN)

(73) Assignee: Tarim University, Ala'er City (CN)

(21) Appl. No.: 17/328,089

(22) Filed: May 24, 2021

(30) Foreign Application Priority Data
Nov. 4, 2020 (CN) 202011214301.6

(51) Int. Cl. C12N 1/20

(52) U.S. Cl. CPC C12N 1/205

(57) ABSTRACT

The present disclosure provides a Brevundimonas strain as an endosymbiont of Verticillium dahliae, which is named Brevundimonas olei dahliae olei (B. olei) and deposited with the accession number of CCTCC NO: M 2020392. Morphological identification and diversity sequencing analysis confirm that the symbiont is B. olei. The effect of B. olei fermentation broth on growth and microsclerotia of Verticillium dahliae is explored through a co-cultivation experiment. Observations through electron microscope find that, with the increase of culture time and fermentation broth concentration, the Verticillium dahliae forms fewer conidia, its hyphae swell, rupture, and become abnormal in shape, and its microsclerotia gradually decrease and even disappear.

### CROSS REFERENCE TO RELATED APPLICATION

This patent application claims the benefit and priority of Chinese Patent Application No. 202011214301.6 filed on Nov. 4, 2020, the disclosure of which is incorporated by reference herein in its entirety as part of the present application.

### TECHNICAL FIELD

The present disclosure relates to the technical field of development and utilization of microbial germplasm resources, specifically relates to a Brevundimonas strain as an endosymbiont of Verticillium dahliae and use thereof.

### BACKGROUND ART

There is a complicated symbiosis relationship between a microorganism and another microorganism, a plant, or animal. Symbionts are common in nature which associate with diverse host types including different animals, plants, corals, macrofungi, aphids, algae and other hosts. They can contribute to or determine certain features of hosts. In addition, the symbionts can fix nitrogen for plants, provide nutrients for plants, participate in synthesis of secondary metabolism in a host, or assist a host in adapting to extreme environments. They can even secrete a variety of active substances to promote or destroy host growth and change the host's phenotype and metabolism.

According to relevant reports, the endosymbiont of a pathogen has a certain relationship with the pathogenicity of the pathogen. Especially, Rhizopus, the pathogen of rice seedling blight, has an endosymbiont Burkholderia cepacia which produces a toxin, causing the rice disease. As for Fusarium, it is the symbiont of Fusarium that exerts an antagonistic effect on Fusarium oxysporum.

Verticillium wilt of cotton is a vascular bundle disease mainly caused by Verticillium dahliae in the soil. It can seriously affect the cotton yield and quality in continuous cropping cotton fields, hindering the healthy and sustainable development of the cotton industry. To date, there has not been any report about whether there is such a symbiotic relationship for Verticillium dahliae that causes cotton Verticillium wilt.

### SUMMARY

In view of this, a technical problem to be solved by the present disclosure is to provide a Brevundimonas strain as an endosymbiont of Verticillium dahliae and use thereof.

To solve the above technical problem, the present disclosure provides the following technical solutions.

A Brevundimonas strain as an endosymbiont of Verticillium dahliae, which is named Brevundimonas olei dahliae olei (B. olei) and deposited with the accession number of CCTCC NO: M 2020392.

A microbial preparation including a sterile culture solution of the B. olei according to claim 1.

The above microbial preparation may be obtained by inoculating the B. olei into a Luria-Bertani (LB) medium, culturing at 37° C. and 180 r·min⁻¹ for 16-24 h, centrifuging at 6,000 r·min⁻¹ for 10 min, taking a supernatant, filtering with a 0.22 μm microporous membrane for 2-3 times to remove microbes, dividing into aliquots and storing in a refrigerator at 4° C. for later use.

During preparation of the above microbial preparation, a mass concentration of the B. olei is 10%-50% after it is inoculated into the LB medium.

Use of the above B. olei in controlling plant diseases caused by fungi, where the fungi are Verticillium dahliae.

Use of the above B. olei in controlling cotton Verticillium wilt.

The technical solutions of the present disclosure have the following beneficial technical effects.

The present disclosure isolates Verticillium dahliae from plants affected by Verticillium wilt in cotton fields in southern Xinjiang, China, and further isolates a symbiont from the Verticillium dahliae through wall breaking and separation, where the symbiont is identified as B. olei. A main objective of the present disclosure is to find out whether the B. olei affects the propagation and pathogenicity of Verticillium dahliae.

Morphological identification and diversity sequencing analysis confirm the symbiont is B. olei.

In vitro experiment: a suspension and a sterile fermentation broth of B. olei are used to explore the effect of the strain on the morphology of Verticillium dahliae and the inhibition rate (IR) through co-cultivation. The study finds that different concentrations of B. olei suspension or fermentation broth can inhibit Verticillium dahliae. After co-cultivation for 15 d, the B. olei suspension with a concentration of 50% can completely inhibit the production of microsclerotia, changing the Verticillium dahliae from the original sclerotia type to the hyphae type; the B. olei fermentation broth can reach a highest IR of 61.30±0.54%.

Pot experiment: The disease index of cotton Verticillium wilt in the treatment groups after 15-30 d from inoculation is quite different. The disease index is 69.75 when the fermentation broth of Verticillium dahliae without symbiont application to cotton seedlings. Compared with other treatment groups, the cotton seedlings after 30 d from B. olei inoculation show no symptoms, and the disease index was 0; namely B. olei is not pathogenic. According to the results of greenhouse pot experiment, B. olei has a relatively desired inhibitory effect on the pathogenicity of cotton Verticillium wilt.

Electron microscope observation: with the increase of culture time and fermentation broth concentration, the Verticillium dahliae forms fewer conidia, its hyphae swell, rupture, and become abnormal in shape, and its microsclerotia gradually decrease and even disappear. The reduction or non-production of Verticillium dahliae microsclerotia will weaken its pathogenicity, alleviating the Verticillium wilt accordingly. This study proves that the symbiotic B. olei has a relatively desired inhibitory activity against Verticillium dahliae.

### BRIEF DESCRIPTION OF THE DRAWINGS

FIG. 1A shows the polymerase chain reaction (PCR) amplification results of 4 strains for Verticillium wilt using fungal primers (M: marker 2KPlusII; lane 1: blank control; lane 2: 10-4; lane 3: 36928; lane 4: 29-16; lane 5: JZ708);

FIG. 1B shows the PCR amplification results of 4 strains for Verticillium wilt using bacterial primers (M: marker 2KPlusII; lane 1: blank control; lane 2: 10-4; lane 3: 36928; lane 4: 29-16; lane 5: JZ708; lane 6: positive control);

FIG. 2 shows species composition and abundance analysis of strains for cotton Verticillium wilt (A: 10-4; B: 29-16; C: 36928; D: JZ708);

FIG. 3 shows morphological identification of symbiont (A: morphology of symbiont colony; B: optical microscope observation; C: scanning electron microscope (SEM) observation; D: Gram staining);

FIG. 4 shows neighbour-joining phylogenetic tree of 4 endosymbiotic strains and their neighboring strains constructed based on the 16S rDNA sequence;

FIG. 5 shows shaking co-cultivation of symbiont and Verticillium dahliae in flasks (growth state of Verticillium dahliae when endosymbiont suspensions diluted to 50%, 20% and 10% concentrations are added to Czapek liquid media and cultivated for 5 d, 10 d and 15 d respectively, in which CK is a control without B. olei suspension);

FIG. 6 shows the colonial morphology of co-cultured symbiont and Verticillium dahliae, where endosymbiont broths diluted to 50%, 20% and 10% concentrations are inoculated into Verticillium dahliae cakes to observe the morphology of Verticillium dahliae, in which A shows top and bottom photos of the solid plates at day 5; B shows the top and bottom photos of the solid plates at day 10; C shows the top and bottom photos of the solid plates at day 15; D shows the statistical diagram of the areas of inhibition zones;

FIG. 7 shows SEM observation of the effect of B. olei on morphology of Verticillium dahliae (A shows the SEM images of co-culture with B. olei, and B shows the SEM images of co-culture with sterile B. olei fermentation broth);

FIG. 8 shows growth of cotton plants in the pot experiment (A: V. dahliae 36928; B: symbiont-free 36928 with symbiont removed by antibiotic; C: V. dahliae 36928+symbiont-free 36928; D: B. olei; E: sterile water as blank control).

### DETAILED DESCRIPTION OF THE EMBODIMENTS

### Example 1: Molecular Biological Identification of Verticillium Wilt Pathogen

DNA extraction of Verticillium wilt pathogen: the hyphae of Verticillium wilt pathogen were snap-freezed with liquid nitrogen to break the walls, ground into powders, and transferred to a 1.5 mL centrifuge tube. 700 μL of CTAB buffer preheated to 65° C. was added and placed in a water bath at 65° C. for 50 min during which the centrifuge tube placed upside down every 10 min to mix the hyphae powders and the buffer thoroughly. The centrifuge was pre-cooled to 4° C. in advance and centrifugation was carried out at 12,000 rpm for 15 min. The centrifuge tube was taken out, and the supernatant was transferred to a new centrifuge tube. 600 μL of DNA extract I was added, mixed well and centrifuged at 12,000 rpm for 15 min. The supernatant was transferred to a new centrifuge tube, and the above step was repeated. The supernatant was transferred to a new centrifuge tube. 600 μL of DNA extract II was added, mixed well and centrifuged at 12,000 rpm for 15 min. The centrifuge tube was taken out, and the supernatant was pipetted into a new centrifuge tube. 600 μL of pre-cooled isopropanol was added, mixed well and placed in the refrigerator at −20° C. for 30 min. The centrifuge tube was taken out and centrifuged at 12,000 rpm for 15 min. The supernatant was discarded. 600 μL of 70% ethanol solution was added to the centrifuge tube to wash the precipitate twice. The precipitate was dried in sterile air, added with 30-50 μL of ddH₂O to dissolve the extracted DNA precipitate, and stored at −20° C. for later use.

Identification of 4 strains for Verticillium wilt with fungal primers (the primers including PCR amplification universal primers ITS1/ITS4 for fungal rDNA-ITS sequence):

Amplification system: 12.5 μL of PCR mix, 1 μL of ITS1 (10 μmoL/L), 1 μL of ITS4 (10 μmoL/L), 1 μL of DNA template (10 ng/μL), and ddH₂O as balance, making up a total volume of 25 μL.

PCR conditions: pre-denaturation at 94° C. for 3 min, denaturation at 94° C. for 30 s, annealing at 55° C. for 30 s, extension at 72° C. for 1 min, a total of 35 cycles; extension at 72° C. for 7 min and storage at 4° C.

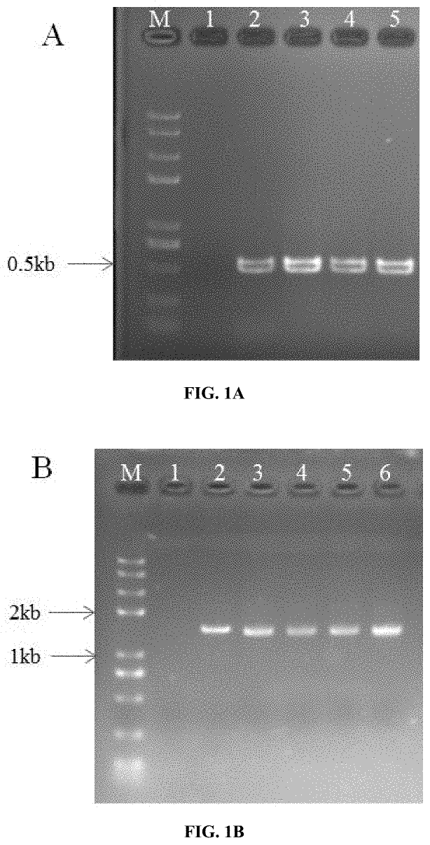

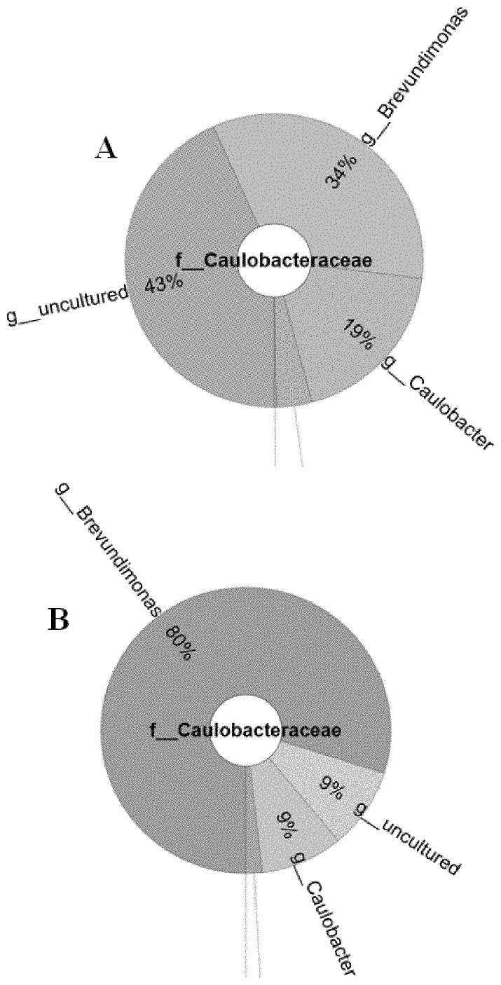

1% agarose gel electrophoresis was used to detect the PCR amplification products. With analysis through gel imaging system and PCR verification, the amplification using primers ITS1/ITS4 yielded a 550 bp single target band (FIG. 1A). With the PCR products sent to Sangon Biotech, Shanghai for sequencing and BLAST homology comparison of the obtained sequences, it was found that the Verticillium wilt strains 10-4, 36928, 29-16, JZ708 were fungi.

Identification of 4 strains for Verticillium wilt with bacterial primers (primers including 16S universal primers, 27F/1492R) Amplification system: 2.5 μL of 10×buffer, 0.5 μL of 10 μM dNTPs, 0.5 μL of 10 μM primer F, 0.5 μL of 10 μM primer R, 0.1 μL of Taq DNA polymerase, 0.5 μL of template DNA, 20.4 μL of ddH₂O.

PCR conditions: pre-denaturation at 94° C. for 4 min, denaturation at 94° C. for 1 min, annealing at 56° C. for 1 min, extension at 72° C. for 2 min, extension at 72° C. for a total of 8 min, a total of 35 cycles; and storage at 4° C.

The PCR products obtained with bacterial primers were detected by 1% agarose gel electrophoresis, obtaining a 1500 bp band of interest (FIG. 1B). This proved that there was a symbiont in the 4 strains for Verticillium wilt. The PCR products were sent for sequence comparison to analyze the taxonomic status.

### Example 2: Detection of Diversity of 4 Strains for Cotton Verticillium Wilt

Microbial total DNA extraction: DNeasy PowerWater Kit was used for extraction, and primers 338F (5′-ACTCCTACGGGAGGCAGCA-3′, SEQ ID NO. 1) and 806R (5′-GGACTACHVGGGTWTCTAAT-3′, SEQ ID NO. 2) were used for PCR amplification of the bacterial 16SrDNA V3-V4 region.

The PCR amplicons were purified with Agencourt AMPure Beads (Beckman Coulter, Indianapolis, Ind.) and quantified with the PicoGreen dsDNA analysis kit. After a separate quantification step, the amplicons were combined in equal amounts, and 2×300 bp sequencing was conducted at both ends using the Illumina MiSeq platform and MiSeq Reagent Kit v3.

Bioinformatic and statistical analysis: the quantitative process for microbial ecology was used to process the sequencing data. Low-quality sequences below were filtered out: sequences with a length of <150 bp, sequences with an average Phred score of <20, sequences containing ambiguous bases, and sequences containing >8 bp of single-nucleotide repeats. Paired-end reads were assembled with FLASH. After chimera detection, UCLUST (Edgar 2010) clustered the remaining high-quality sequences into operational taxonomic unit (OTU) with 97% sequence identity. Default parameters were used to select a representative sequence from each OTU.

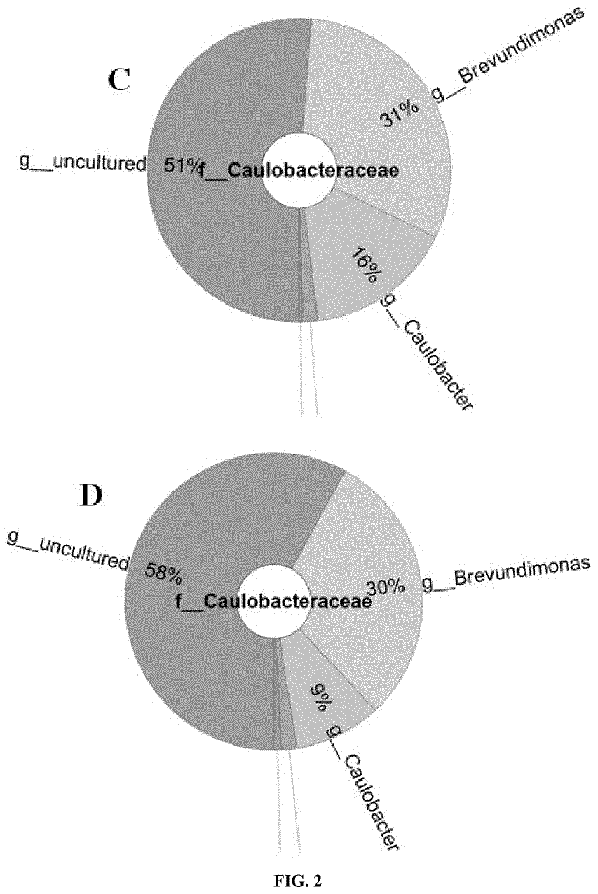

Sequence data analysis was mainly performed using QIIME and R software packages (v3.2.0). OTU-level alpha diversity indices were evaluated with, for example, Chaol abundance estimator and ACE indicator (coverage estimator based on abundance), where the OTU table in QIIME was used to calculate Shannon diversity index and Simpson index. A rank abundance curve was generated based on OTU level to compare the abundance and evenness of OTU between samples. Beta diversity analysis was performed using the UniFrac distance metric to investigate the structural changes of microbial community between samples. Principal component analysis (PCA) (Ramette 2007) was also performed based on the composition profile at the genus level. PERMANOVA (variance multivariate analysis) and ANOSIM (analysis of similarity) were used to evaluate the microbial community structure differentiation between groups. Results showed that Brevundimonas was detected in the 4 samples with the following content in each sample: A (10-4): 34%, B (29-16): 80%, C (36928): 31%, and D (JZ708): 30% (FIG. 2). Sample B with the highest content was of hyphae type without microsclerotia, and the other 3 samples belonged to the sclerotia type. It can be inferred that the Brevundimonas contents in the Verticillium wilt strains were related to the hyphae and sclerotia of the strains, and the content was relatively high in the Verticillium wilt strain of hyphae type.

### Example 3: Isolation and Identification of 4 Endosymbiotic Bacteria for Cotton Verticillium Wilt

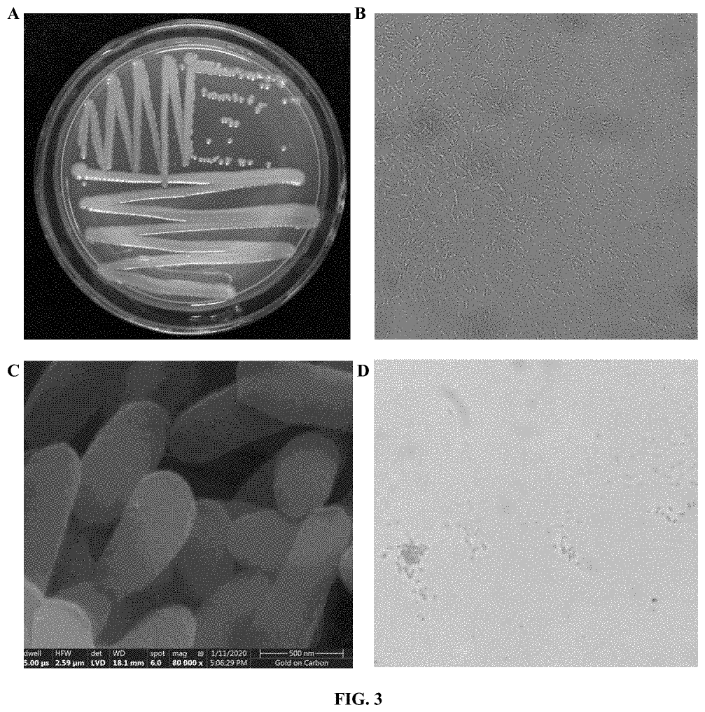

Isolation and morphological identification of endosymbiotic bacteria: the cotton Verticillium wilt strains were cultivated for 7-10 d, and taken in an amount of about a match head in size by an inoculating needle through scraping respectively. According to a general mechanical breaking method, each strain was snap-freezed with liquid nitrogen and ground in a frozen mortar to break the walls. Gradient dilution was carried out. 30 μL of microbial suspensions at gradient concentrations was plated onto LA culture medium and cultured at 37° C. for 16-24 h. The isolated symbiotic bacterial strain was preliminarily identified through morphological features, and grown on LB medium. The symbiotic bacteria had milky yellow colonies with a raised center and a shiny and sticky surface (shown in FIG. 3A).

An inoculating needle was used to pick the colonies and place them on a glass slide. 2 μL of sterile water was added to disperse the colonies. The microbes were observed under a 40× microscope as short rods (FIG. 3B). The endosymbiont suspension cultured in LB medium for 16 h was taken and centrifuged. The supernatant was discarded and the bacteria were collected. Pre-fixation was carried out by adding 500-1,000 μL of 5% glutaraldehyde fixative solution to the endosymbiont. Fixation was carried out overnight at 4° C. Gradient dehydration was carried out. 300-500 μL of 30% ethanol was added, aspirated and mixed well to dehydrate for 15-20 min. After centrifugation, the supernatant was discarded. The above steps were repeated with 50% ethanol, 70% ethanol, 90% ethanol, and 95% ethanol for dehydration. 100% ethanol was used to carry out dehydration twice, with 15-20 min each time. After centrifugation, the supernatant was discarded. 100% ethanol:100% acetone (1:1) was used to perform dehydration for 15-20 min. After centrifugation, the supernatant was discarded. 100% acetone was used to carry out dehydration twice, with 15-20 min each time. After centrifugation, the supernatant was discarded. A critical point dryer was used for drying and dehydration. Sputter coating was carried out with an ion sputterer. SEM observations showed that the strain took the shape of short rod (FIG. 3C). Gram staining: a smear was fixed, stained with ammonium oxalate crystal violet for 1 min, rinsed with distilled water, covered with iodine solution, dyed for 1 min, subjected to 95% decolorization for about 20 s, rinsed with water, dyed with safranin staining solution for 1 min and rinsed with distilled water. After drying, microscopic examination showed red microbes, that is, the Gram staining was negative (FIG. 3D).

Molecular biological identification of endosymbiotic bacteria: the bacteria were collected by centrifugation at 12,000 rpm for 10 min, and the supernatant was discarded. 150 μL of solution I was added, and mixed well with the bacteria. 300 μL of solution II was added and shaken slightly. 230 μL of solution III was added, shaken and mixed well. Milky white flocs appeared. The supernatant was taken into a new EP tube, added with 5 μL of RNase and 100 μL of phenol:chloroform:isoamyl alcohol (25:24:1), and centrifuged at 12,000 rpm for 10 min. The supernatant was taken, added with 100 μL of chloroform, shaken and mixed uniformly, and centrifuged at 12,000 rpm for 10 min. The supernatant was taken into a new EP tube, added with an equal volume of isopropanol, and allowed to stand still at −20° C. for 30 min. Centrifugation was carried out at 12,000 rpm for 10 min. The supernatant was discarded. The precipitate was washed twice with 70% ethanol. After drying, 30 μL of ddH₂O was added and stored at −20° C. for later use.

PCR confirmation of endosymbiont: the amplification system included 2.5 μL of 10×buffer, 0.5 μL of 10 μM dNTPs, 0.5 μL of 10 μM primer F, 0.5 μL of 10 μM primer R, 0.1 μL of Taq DNA polymerase, 0.5 μL of template DNA, and 20.4 μL of ddH₂O.

PCR conditions: pre-denaturation at 94° C. for 4 min, denaturation at 94° C. for 1 min, annealing at 56° C. for 1 min, extension at 72° C. for 2 min, extension at 72° C. for a total of 8 min, a total of 35 cycles; and storage at 4° C.

When PCR amplification was completed, electrophoresis detection was carried out. PCR products were detected through a gel imaging system. Amplification with universal primers 27F/1492R for 16S rDNA sequence yielded the target band with a size of 1,500 bp.

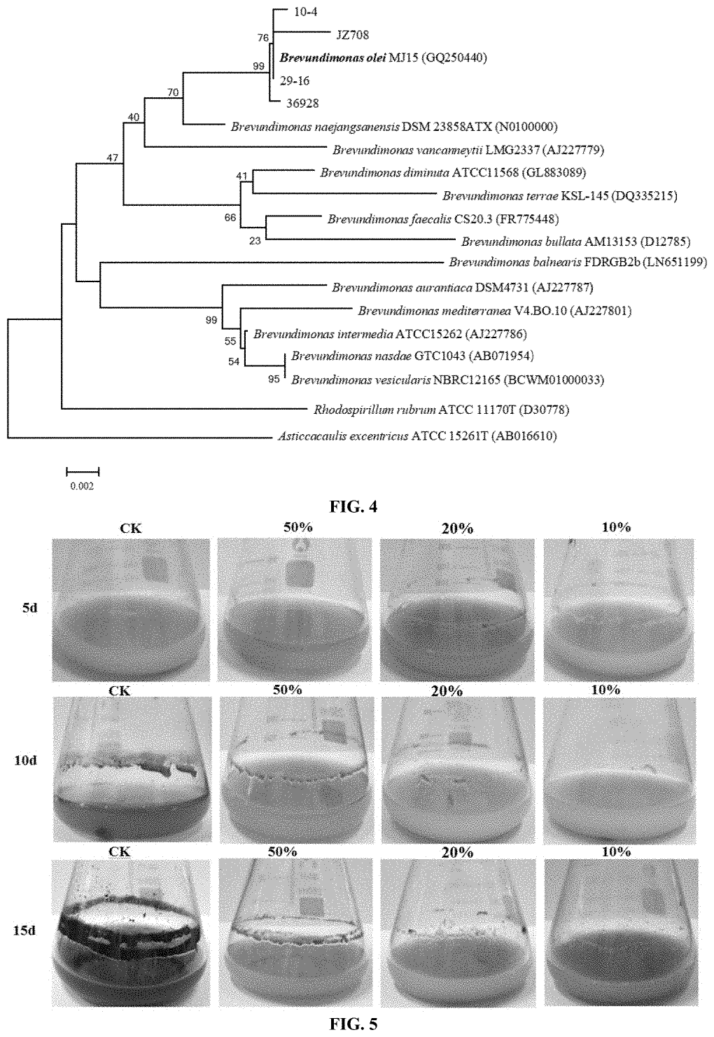

After sequencing, Blast comparison was carried out in NCBI, followed by clustering analysis using the neighbor-joining method with the MEGA software, and construction of a phylogenetic tree (FIG. 4). Based on the phylogenetic tree, the 4 symbiotic strains in Verticillium dahliae were preliminarily determined as B. olei.

B. olei, the symbiont in Verticillium dahliae, was deposited in the China Center for Type Culture Collection (CCTCC) in Wuhan University, Wuhan City, Hubei Province (address: Collection Center of Wuhan University, No. 299, Bayi Road, Wuchang District, Wuhan City, Hubei Province; zip code: 430072) on Aug. 3, 2020 with the accession number of CCTCCNO: M2020392.

### Example 4: Co-Cultivation of B. olei and Verticillium dahliae and Inhibitory Effect

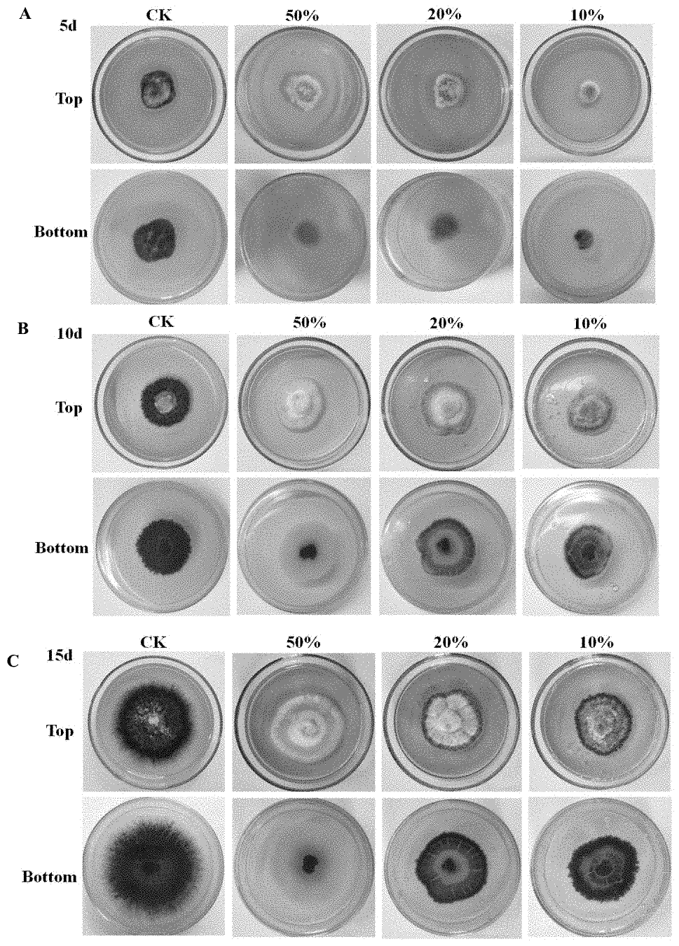

The activated Verticillium dahliae in a PDA plate was beaten into 7 mm cakes (2-3 pieces), inoculated into 100 mL of Czapek-Dox culture medium, and cultured at 120 r·min⁻¹ and 25° C. for 3 d. 25 mL of endosymbiont suspensions diluted to 50%, 20% and 10% concentrations were respectively added to 100 mL of Czapek liquid medium. Co-cultured was carried out for 15 d. A control without B. olei was used. The growth status of Verticillium dahliae in shake flasks with different treatments was observed. Results showed that the growth status of Verticillium dahliae co-cultured in shake flasks differed significantly with the concentration of B. olei. With the increase of culture time, the color of the liquid in shake flasks of the treatment groups and the control group changed. Compared with the control group, the treatment groups added with B. olei suspensions showed lighter color. After co-cultivation for 15 d, the 3 treatment groups with different B. olei concentrations were observed. Melanin production was most obvious in shake flasks added with 10% B. olei suspension, followed by those added with 20% B. olei suspension, while there was no significant color change in shake flasks added with 50% B. olei suspension (FIG. 5). The treatment group added with a greater concentration of B. olei suspension produced less melanin in liquid within the shake flask.

An endosymbiotic bacteria fermentation broth was prepared. The endobiont was inoculated in LB medium, cultured at 37° C. and 180 r·min⁻¹ for 16-24 h and centrifuged (6,000 r·min⁻¹) for 20 min. The supernatant was taken, filtered with a microporous membrane (0.22 μm) for 2-3 times to remove microbes, divided into aliquots, and stored in a refrigerator at 4° C. for later use.

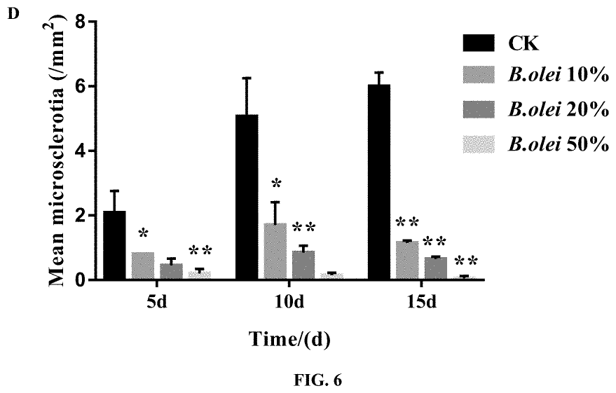

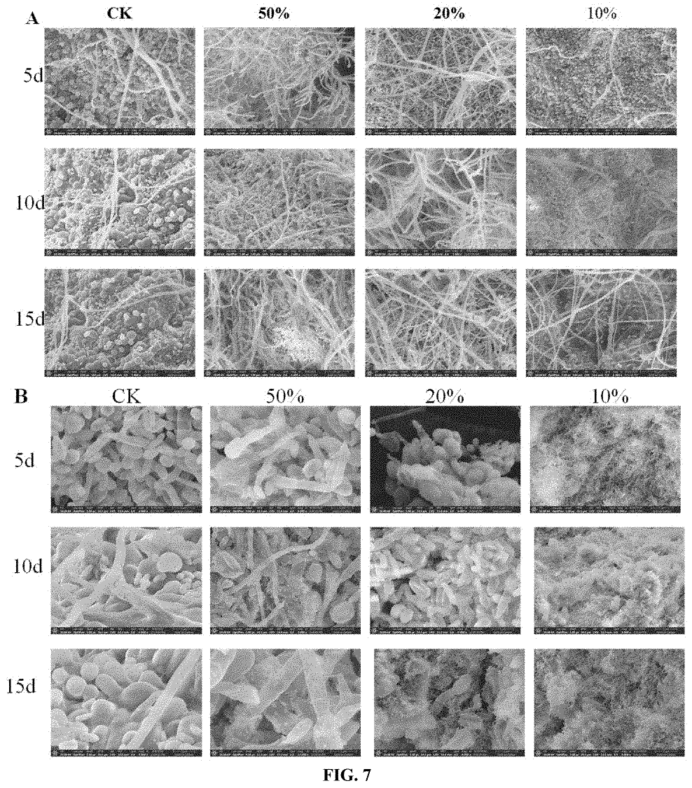

The effect of solid plate co-cultivation on the growth morphology of Verticillium dahliae and inhibitory effect were evaluated below. Treatment group (TM): 100 mL of sterilized PDA medium was cooled to about 45° C., and added with 25 mL of B. olei fermentation broth diluted to 50%, 20% and 10% concentrations respectively. Control group (CK): the same medium as the treatment group was used but without the fermentation broth. The media in both groups were shaken, mixed uniformly, and poured to plates to form PDA plates. After the plates were solidified for 12 h, the cotton Verticillium wilt strain cake was inoculated to centers of the PDA plates of the control group and the treatment group respectively. Pictures were taken every 5 d to observe the morphology of Verticillium dahliae. The crossing method was used to measure the colony size for different treatments, and the IR of the fermentation broth was calculated based on the colony diameter: IR (%)=[(diameter of control colony−diameter of treated colony)/(diameter of control colony−7)]×100. In three different culture periods, the B. olei fermentation broths at different concentrations had an effect on the morphology of Verticillium dahliae. Compared with the control group, the treatment groups had vigorously grown hyphae. When the fermentation broth with a 50% concentration was used for co-cultivation for 10 d and 15 d, the Verticillium dahliae colonies produced no microsclerotia (FIGS. 6B-C), changing from the original sclerotia type to the hyphae type. The number of microsclerotia in the treatment groups co-cultured with B. olei fermentation broth was always smaller than that in the control group (FIG. 6D). SEM observations showed that, with the increase of culture time and B. olei concentration, the Verticillium dahliae formed decreased number of conidia, its hyphae swelled, ruptured, and became abnormal in shape, and its microsclerotia gradually decreased and even disappeared (FIG. 7). For co-cultivation with B. olei suspensions, B. olei with a short rod shape was observed to attach to the surface of Verticillium dahliae mycelium (FIG. 7B).

All the B. olei fermentation broths at different concentrations inhibited the growth of Verticillium dahliae, with quite different IRs among different treatments (Table 1). The 50% fermentation broth showed a relatively desired inhibitory effect, with IR reaching 61.30±0.54% after co-cultivation for 15 d. When the fermentation broths had a concentration of 10% or 20%, their IRs were lower than that of the 50% B. olei fermentation broth after co-cultivation for 5 d, 10 d and 15 d.

TABLE 1
IRs of B. olei fermentation broth at different concentrations
on Verticillium dahliae
Dilution

| | 50% | 20% | 10% |
|---|---|---|---|
| Culture time (d) | IR | IR | IR |
| 5 | 38.80 ± 1.23 | 37.00 ± 0.47 | 29.90 ± 0.35 |
| 10 | 54.40 ± 0.73 | 40.70 ± 0.75 | 31.70 ± 0.99 |
| 15 | 61.30 ± 0.54 | 44.80 ± 4.51 | 32.40 ± 0.43 |

### Example 5: Greenhouse Pot Experiment

Cotton seed treatment: 70% ethanol was used to remove the cotton seed coatings. The seeds were soaked in warm water at 55° C. for 30 min, then at room temperature for 8 h and drained. Germination was accelerated at 25° C. When the seeds showed white contents, they were sown. Cotton seedling cultivation:mixed nutrient soil (flower nutrient soil:natural field soil of 1:1) was sterilized, and 3-5 seeds were sown for each flowerpot. Preparation of microbial suspensions for the treatment groups: a. V. dahliae 36928, b. symbiont-free 36928 (symbiont removed by antibiotic), c. V. dahliae 36928+symbiont-free 36928, d. B. olei, e. sterile water as blank control. The concentration of microbial suspension was 1×10⁹ cfu/mL. A root-watering method was used to inoculate the pathogen. When cotton seedlings had two true leaves, the pathogen was inoculated in an amount of 10 mL per pot. After inoculation, watering was carried out every 2 days and the growth status was observed. At day 7 after inoculation, there was sporadic onset of disease and at day 15, the disease was widespread. After that, the disease was investigated using a 5-level grading method with cotton seedling leaves. The disease-level grading standards were: level 0: healthy plants; level 1: 1-2 cotyledons showing symptoms; level 2: 1-2 true leaves showing obvious symptoms; level 3: 3 or more true leaves showing symptoms; level 4: dead meristem region of plant or the whole plant dead. The disease index was summarized. According to the disease index of Verticillium dahliae on cotton plants, the pathogens were divided into 3 pathogenic types: type I was highly pathogenic with an average disease index of 35.1-100; type II was moderately pathogenic with an average disease index of 20.1-35.0; type III was weakly pathogenic with an average disease index of 0.1-20.0.

$$Disease index = x = \frac{\begin{matrix}Σ ( number of diseased leaf at any level \times \\ numerical number of the relative level )\end{matrix}}{\begin{matrix}total number of investigated leaves \times \\ numerical value of the highest level\end{matrix}} \times 100$$

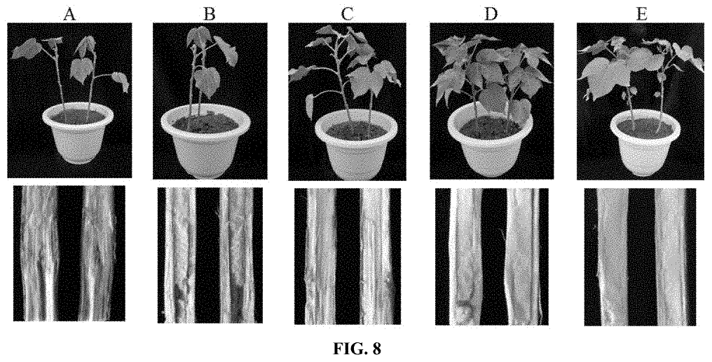

In the pot experiment, all the groups treated with the microbial suspensions had different degrees of disease, where the cotton plants wilted and burned, with leaves fell off. The stalks of cotton plants were dissected, and it was found that the vascular bundles of cotton stalks with symbiont-free 36928 microbial suspension had serious dark brown lesions (FIG. 8B), while those treated with B. olei suspension or sterile water had no obvious symptoms (FIG. 8D-E). When cotton seedlings were grown for 30 d, the disease index with treatment of symbiont-free 36928 and V. dahliae 36928 was 69.75 and 53.71 respectively, both were highly pathogenic. From the appearance and disease index of the cotton plant, it can be seen that the symbiont-free 36928 had higher pathogenicity, while B. olei was not pathogenic (Table 2).

TABLE 2
Disease index in pot experiment

| | Disease index | | |
|---|---|---|---|
| Different treatments | 15 d | 20 d | 30 d |
| V. dahliae36928 | 36.40 ± 9.51 | 44.78 ± 8.23 | 53.71 ± 8.09 |
| symbiont-free36928 | 51.25 ± 8.75 | 55.25 ± 9.93 | 69.75 ± 7.82 |
| V. dahliae36928 + | 17.71 ± 7.20 | 28.13 ± 8.73 | 33.75 ± 9.76 |
| symbiont-free36928; | | | |
| B. olei | 0 | 0 | 0 |
| CK | — | — | — |

Note:
the data in the table was mean ± standard deviation

It is apparent that the above embodiments are merely listed for clear description, and are not intended to limit the implementations. Those of ordinary skill in the art may make modifications or variations in other forms based on the above description. There is no need and no way to exhaust all of the implementations. However, obvious changes or variations thus introduced still fall within the protection scope of the claims in the present patent application.

The attached text file “sequence_listing.txt” created Apr. 11, 2020 that is 1000 bytes is hereby incorporated by reference.

Sequence Listing
Primer used 338F
SEQ ID NO.: 1
5′-ACTCCTACGGGAGGCAGCA-3′
Primer used 806R
SEQ ID NO.: 2
GGACTACHVGGGTWTCTAAT-3′

What is claimed is:

1. A microbial preparation comprising a sterile culture solution of a Brevundimonas strain as an endosymbiont of Verticillium dahliae, which is named Brevundimonas olei dahliae olei (B. olei) and deposited with the accession number of CCTCC NO: M 2020392.

2. The microbial preparation according to claim 1, wherein the microbial preparation is obtained by inoculating the B. olei into a Luria-Bertani (LB) medium, culturing at 37° C. and 180 r·min⁻¹ for 16-24 h, centrifuging at 6,000 r·min⁻¹ for 10 min, taking a supernatant, filtering with a 0.22 μm microporous membrane for 2-3 times to remove microbes, dividing into aliquots and storing in a refrigerator at 4° C. for later use.

3. The microbial preparation according to claim 2, wherein a mass concentration of the B. olei is 10%-50% after the B. olei is inoculated into the LB medium.
